(19) United States
(12) Patent Application Publication
(10) Pub. No.: US 2010/0120157 A1
(43) Pub. Date: May 13, 2010

(54) Methods of Using Sulfur Nucleophiles as Improved Alternatives to Sodium Bisulfite for Methylated DNA Analysis

(75) Inventor: Gerald Zon, San Carlos, CA (US)

Correspondence Address: LIFE TECHNOLOGIES CORPORATION;C/O INTELLEVATE, P.O. BOX 52050, MINNEAPOLIS, MN, 55402, US

(73) Assignee: LIFE TECHNOLOGIES CORPORATION, Carlsbad, CA (US)

(21) Appl. No.: 12/557,477

(22) Filed: Sep. 10, 2009

Related U.S. Application Data
(63) Continuation of application No. 11/231,480, filed on Sep. 21, 2005, now abandoned.
(60) Provisional application No. 60/611,779, filed on Sep. 21, 2004.

(51) Int. Cl. G01N 23/00; C07H 1/00; G01N 33/53

(52) U.S. Cl. 436/57; 536/55.3; 436/94

(57) ABSTRACT

The invention provides for the use of sulfur nucleophiles in analyzing methylated DNA and novel sulfur nucleophiles suitable for such us.

### CROSS REFERENCE TO RELATED APPLICATIONS

The present application claims priority under 35 U.S.C. §119(e) to Provisional Application Ser. No. 60/611,779, Filed: Sep. 21, 2004. Such application is hereby incorporated by reference in its entirety.

### FIELD OF INVENTION

The invention relates generally to sulfur nucleophiles and methods of using them for analysis of methylated DNA.

### BACKGROUND OF THE INVENTION

Assessment of the methylation of DNA is useful in many research, diagnostic, medical, forensic, and industrial fields. Particularly, methylation of cytosine in genomic DNA has been correlated with lack of gene expression, and in some instances can be indicative of early and frequent alterations found in some cancers. Thus, the ability to assess the methylation status of DNA is significant.

Key to this assessment is converting cytosine to uracil. One basic method for such conversion, employing sodium bisulfite (NaHSO₃), is well known. Over the years, the method has been improved in attempts to overcome disadvantages that include tedious procedures, lengthy reaction times, and DNA degradation. The most commonly used protocol is taught by J. Herman, Proc. Natl. Acad. Sci. 93, 9821-26 (1996), incorporated herein by reference in its entirety. This method involves denaturation, reaction with sodium bisulfite in the presence of hydroquinone, and subsequent completion of the modification by treatment with NaOH. Despite the attempts to improve the protocol, it is still required to pre-denature the genomic DNA (gDNA) to single stranded DNA (ssDNA), prepare fresh solutions of sodium bisulfite, typically about 3M, and include an antioxidant (e.g., hydroquinone). The protocol also involves long reaction times and tedious clean-up procedures.

In addition, the currently employed sodium bisulfite protocols are plagued by reports of incomplete conversion, irreproducible results, and other problems. In some cases, the reaction can result in significant DNA degradation (reportedly as high as 96%), making it difficult to obtain enough sample for further analysis. See. S. J. Clark et al. Nucleic Acid Research 2001, 29 no. 13, e65.

Other methods exist to assess methylation status. Many of these methods use labeling technology. For example, radio-labeled samples can be compared to internal standards by GC-MS (P. F. Crain and J. A. McCloskey. Anal. Biochem. (1983) 132, 124-131). Fluorescent or chemiluminescent moieties may be used to assess methylation status through optical detection means. These usually require sophisticated and expensive HPLC or CE equipment operated by experts (M. Wirtz et al. Electrophoresis (2004) 25, 839-845; D. Stach et al. Nucleic Acids res. (2003) 31, E2.). One current approach, useful in analyzing CpG islands, is restriction landmark genome scanning (RLGS), which is based on digestion of DNA with methylation-sensitive restriction enzymes, radiolabeling and then 2D-gel separation (D. J. Smiraglia et al. Genomics (1999) 58, 254-262.). RLGS is therefore limited to only those CpG islands which contain sites compatible with available restriction enzymes.

Given the importance of assessment of DNA methylation, it can be seen that there is a need for improved methodologies for conversion of cytosine to uracil and for assessing the methylation status of DNA.

### SUMMARY OF THE INVENTION

In some embodiments, a method for converting cytosine to uracil in a nucleic acid comprises the steps of:

providing a nucleic acid comprising at least one cytosine nucleobase; and

reacting said nucleic acid with a nucleophilic organo-sulfur compound.

In some embodiments, a nucleophilic organo-sulfur compound Formula I:

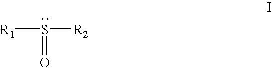

wherein R₁ and R₂ are each independently selected from the group consisting of hydroxyl, alkyl, aryl, amino, alkoxy, and aryloxy, each of which may be optionally substituted; and

or R₁ and R₂ can be concatenated to form a 4-8 membered ring optionally having 1 or 2 additional hetero ring atoms selected from N, S, and O, wherein said ring can be optionally substituted with one or more substituents;

or a salt thereof

is reacted with a nucleic acid comprising at least one cytosine nucleobase, prior to assessment of methylation status.

In some embodiments, the methods herein are carried out with a salt of formula I where one or both of R₁ and R₂ forms an ionic bond (or salt pair) with a cation selected from lithium, sodium, magnesium and ammonium. In such embodiments, one or both R₁ and R₂ may comprise(s) an anionic group capable of forming such ionic bond or salt pair.

In some embodiments, a method for assessing the methylation status of cytosine comprises the steps of:

providing a sample nucleic acid comprising at least one cytosine nucleobase of unknown methylation status; and

reacting said nucleic acid with a nucleophilic organo-sulfur compound comprising a radio-labeled substituent.

In some embodiments, the nucleophilic organo-sulfur compound is a compound of formula I:

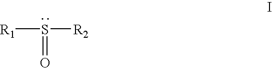

wherein R₁ and R₂ are each independently selected from the group consisting of hydroxyl, alkyl, aryl, amino, alkoxy, and aryloxy, and a radiolabel substituent, wherein each of said alkyl, aryl, amino, alkoxy, and aryloxy can be optionally substituted;

wherein at least one of R₁ and R₂ comprises a radio-labeled substituent;

or a salt thereof.

In some embodiments, such methods further provide the steps of:

providing a control nucleic acid comprising at least one cytosine nucleobase of known non-methylated status;

reacting said nucleic acid with the same said nucleophilic organo-sulfur compound; and

comparing the level of radioactivity of the sample and control to determine the relative content of methylated cytosine in the sample based on the rates of reaction of the methylated cytosine and unmethylated cytosine.

In some embodiments, a method for assessing the methylation status of cytosine comprises the steps of:

providing a sample nucleic acid comprising at least one cytosine nucleobase of unknown methylation status; and

reacting said nucleic acid with a nucleophilic organo-sulfur compound comprising a fluorescent or chemiluminescent moiety.

In some embodiments, the nucleophilic organo-sulfur compound comprising a fluorescent or chemiluminescent moiety is a compound of formula I:

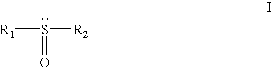

wherein R₁ and R₂ are each independently selected from the group consisting of hydroxyl, alkyl, aryl, amino, alkoxy, and aryloxy, and a radiolabel substituent, wherein each of said alkyl, aryl, amino, alkoxy, and aryloxy can be optionally substituted;

wherein at least one of R₁ and R₂ comprises a fluorescent or chemiluminescent moiety;

or a salt thereof.

### DETAILED DESCRIPTION

It is to be understood that both the foregoing general description and the following detailed description are exemplary and explanatory only and are not restrictive of the invention. In this application, the use of the singular includes the plural unless specifically stated otherwise. In this application, the use of “or” means “and/or” unless stated otherwise. The use of the term “comprising,” as well as other forms, such as “comprises” and “comprise,” will be considered inclusive, in that the term “comprising” leaves open the possibility of including additional elements. Furthermore, the use of the terms “including” or “having”, as well as other forms, such as “includes”, “has”, “included”, and “have” is not intended to be limiting. Also, terms such as “element” or “component” encompass both elements and components comprising one unit and elements and components that comprise more than one subunit unless specifically stated otherwise.

The section headings used herein are for organizational purposes only and are not to be construed as limiting the subject matter described.

The term “alkyl” refers to straight and branch chain hydrocarbon groups, such as, but not limited to, methyl, ethyl, propyl, butyl, pentyl, hexyl, heptyl, octyl, nonyl, decyl, undecyl, dodecyl and the like. The term also includes branched chain isomers of straight chain alkyl groups, including but not limited to, the following which are provided by way of example: —CH(CH₃)₂, —CH(CH₃)(CH₂CH₃), —CH(CH₂CH₃)₂, —C(CH₃)₃, —C(CH₂CH₃)₃, —CH₂CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH₃)CH(CH₃)(CH₂CH₃), —CH₂CH₂CH(CH₃)₂, —CH₂CH₂CH(CH₃)(CH₂CH₃), —CH₂CH₂CH(CH₂CH₃)₂, —CH₂CH₂C(CH₃)₃, —CH₂CH₂C(CH₂CH₃)₃, —CH(CH₃)CH₂CH(CH₃)₂, —CH(CH₃)CH(CH₃)CH(CH₃)CH(CH₃)₂, —CH(CH₂CH₃)CH(CH₃)CH(CH₃)(CH₂CH₃), and others. The term also includes cyclic alkyl groups such as cyclopropyl, cyclobutyl, cyclopentyl, cyclohexyl, cycloheptyl, and cyclooctyl and such rings substituted with straight and branched chain alkyl groups as defined above. Thus alkyl groups include primary alkyl groups, secondary alkyl groups, and tertiary alkyl groups. Preferred alkyl groups include straight and branched chain alkyl groups and cyclic alkyl groups having 1 to 12 carbon atoms.

The term “alkoxy” refers to a group of formula —O-alkyl, where alkyl is as defined above. Examples include but are not limited to —OMe, —O Et, and the like.

The term “aryl” is intended to denote a radical derived from a compound that contains at least one aromatic ring. Thus, aryl groups include, but are not limited to, groups such as phenyl and biphenyl, and groups containing condensed rings such as naphthalene and anthracene. A preferred unsubstituted aryl group is phenyl.

The term “amino” refers to a nitrogen having two substituents. The substituents are independently selected and include, but are not limited to, hydrogen, hydroxyl, alkyl, aryl, etc. and may be optionally substituted. Most preferred are hydrogen, methyl, ethyl, propyl, isopropyl, 2-hydroxyethyl, and 2-methoxyethyl.

The term “aryloxy” refers to a group of formula —O-aryl, where aryl is as defined above. One non-limiting example of an aryloxy group is a phenoxy group; i.e., a group of formula —OPh where Ph is phenyl.

The term “bisulfite ion,” as used herein, has its accustomed meaning of HSO₃—. Typically, bisulfite is used as an aqueous solution of a bisulfite salt, for example magnesium bisulfite, which has the formula Mg(HSO₃)₂, and sodium bisulfite, which has the formula NaHSO₃.

The phrase “optionally substituted” refers to groups in which one or more hydrogen atoms have been replaced by a non-hydrogen substituent group. Such groups include, but are not limited to, halogen atoms such as F, Cl, Br, and I; hydroxyl groups, alkyl groups, alkenyl groups, alkynyl groups, aryl groups, alkoxy groups, aryloxy groups, ester groups; thiol groups, alkyl and aryl sulfide groups, sulfone groups, sulfonyl groups, sulfoxide groups, amines, amides, alkylamines, dialkylamines, arylamines, alkylarylamines, diarylamines, N-oxides, imides, enamines, trialkylsilyl groups, dialkylarylsilyl groups, alkyldiarylsilyl groups, and triarylsilyl groups.

The term “PCR” is intended to denote polymerase chain reaction, as is well known in the art. The term “MSP” denotes methylation specific PCR, such as described by J. Herman, Proc. Natl. Acad. Sci. 93, 9821-26 (1996), incorporated herein by reference in its entirety.

The term “nucleic acid sample” is intended to denote a sample (e.g., a composition, mixture, suspension or solution) that contains at least one nucleic acid.

As used herein, the term “nucleic acid” includes nucleobase-containing polymeric compounds, including naturally occurring and non-naturally occurring forms thereof, for example and without limitation, genomic DNA, cDNA, hnRNA, mRNA, rRNA, tRNA, fragmented nucleic acids, nucleic acids obtained from subcellular organelles such as mitochondria or chloroplasts, and nucleic acids obtained from microorganisms, or DNA or RNA viruses that may be present on or in a biological sample.

As used herein, the term “gDNA” refers to genomic DNA.

“Fluorescent moiety,” as used herein, means a moiety that fluoresces (i.e. emits light of a certain wavelength) when exposed to radiation. Examples of such moieties include but are not limited to 6-carboxyfluorescein or 6-carboxytetramethylrhodamine.

“Chemiluminescent moiety” means a moiety that allows chemiluminescent activity (i.e. generation of light by chemical reaction) to be detected by optical means. Examples of such moieties include but are not limited to acridinium esters and derivatives thereof.

“Nucleophilic organo-sulfur compound” as used herein refers to those compounds having a lone pair of electrons at sulfur. Preferred nucleophilic organo-sulfur compounds are substituted derivatives of sulfinic acid. Most preferred are those of formula I, discussed below.

There are a wide variety of compounds which can formally be viewed as derivatives of the HO—S(:)(O)—OH moiety that preserve the nucleophilic lone-pair of electrons (:) at sulfur. While not wishing to be bound by a particular theory, it is believed that this nucleophilic lone-pair of electrons at sulfur modulates the specificity and rate of the reversible adduct formation with cytosine which in turn influences the subsequent irreversible hydrolysis to generate uracil. Consequently, certain derivatives of HO—S(:)(O)—OH may have desirable features with regard to cytosine-to-uracil conversion prior to analyses of methylated DNA.

The nucleophilicity of the sulfur compounds has been indicated as the basis of attack of sulfur at carbon in an aromatic ring (A. Ulman and E. Urankar, J. Org. Chem. (1989) 54, 4691-4692), at an unsaturated (acetylenic) carbon (T. Kataoka et al. Phosphorus, Sulfur and Silicon and the Related Elements (1998) 136/138, 497-500), and at the carbon-carbon double bond in acrylonitrile (I. V. Bodrikov et al. Z. Org. Khim. (1985) 21, 1017-1022). Each supports the present invention that the nucleophilicity of the sulfur compounds provides the basis for reaction with cytosine to yield uracil. None, however, teaches the conversion of cytosine to uracil.

### General Preparation of Nucleophilic Organo-Sulfur Compounds

Mono-substituted organo-sulfur nucleophiles are made by replacing one —OH moiety attached to sulfur, S, with alkyl, aryl, amino, alkoxy, or aryloxy groups, which may in turn be substituted with various other groups. The remaining —OH group may be used to form a salt, preferably lithium or magnesium, more preferably sodium, and therefore be ionic.

Bis-substituted, non-ionic compounds may also be formed where both —OH groups are replaced, independently, with alkyl, aryl, amino, alkoxy, or aryloxy groups, which in turn may be substituted with various other groups.

A variety of mono-substituted and bis-substituted derivatives of HO—S(:)(O)—OH, including sodium, lithium, and magnesium salts thereof, are known in the art. Form example, derivatives of HO—S(:)(O)—OH are found in FR 2,288,086, hereby incorporated by reference. FR 2,288,086 also discloses sulfinic acids where one —OH is replaced with an alkyl group and sulfinic esters where one OH is replaced by an alkyl group and the other by an alkoxy group are disclosed.

Dialkyl sulfates where both —OH moieties of the bisulfite are replaced with alkoxy groups are disclosed in M. Mikolyczyk and coworkers in Tetrahedron (1988) 44 (16) 5243, which is hereby incorporated in its entirety by reference.

### Exemplary Sulfur Nucleophiles

Some embodiments of the methods of the present invention employ sulfur nucleophiles according to formula I:

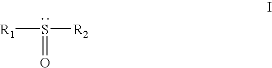

wherein R₁ is selected from the group consisting of hydroxyl, alkyl (R), aryl (Ar), amino (NR₃R₄), alkoxy (OR₅), and aryloxy (ArO), each of which may be optionally substituted, and each of which optionally may be labeled with one of a radio-marker, a fluorescent moiety, and a chemiluminescent moiety;

wherein R₂ is selected from the group consisting of hydroxyl, alkyl (R), aryl (Ar), amino (NR₃R₄), alkoxy (OR₅), and aryloxy (ArO), each of which may be optionally substituted and each of which optionally may be labeled with one of a radio-marker, a fluorescent moiety, and a chemiluminescent moiety;

or, wherein R₁ and R₂ are concatenated to form a 4-8 membered ring optionally having 1-2 additional hetero ring atoms selected from N, S, and O, and optionally substituted with one or more substituents;

R₃ and R₄ are each independently selected from the group consisting of alkyl, substituted alkyl, aryl, and substituted aryl;

R₅ is an alkyl or substituted alkyl;

or, a salt thereof, such as a lithium, sodium, ammonium or magnesium salt wherein one of R₁ and R₂ forms an ionic bond with a halide ion.

Some representative mono-substituted sulfur nucleophiles of formula I where R₂ is —OH, and salts thereof, are listed in table 1:

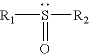

| Compound | Structure (R₂—S(O)—R₁) |
|---|---|
| sulfurous acid, monomethyl ester | HO—S(O)—OCH₃ |
| (monomethyl sulfite) | |
| sulfurous acid, monomethyl ester, | Li•O—S(O)—OCH₃ |
| lithium salt (lithium methyl sulfite) | |
| sulfurous acid, monomethyl ester, | Na•O—S(O)—OCH₃ |
| sodium salt (methyl sodium sulfite) | |
| sulfurous acid, monomethyl ester, | 1/2 Mg•O—S(O)—OCH₃ |
| magnesium salt | |
| sulfurous acid, monoethyl ester | HO—S(O)—OCH₂CH₃ |
| (monoethyl sulfite) | |
| sulfurous acid monoethyl ester, | Li•O—S(O)—OCH₂CH₃ |
| lithium salt | |
| sulfurous acid monoethyl ester, | Na•O—S(O)—OCH₂CH₃ |
| sodium salt (ethyl sodium sulfite; | |
| sodium ethyl sulfite) | |
| sulfurous acid monoethyl ester, | 1/2 Mg•O—S(O)—OCH₂CH₃ |
| magnesium salt | |
| sulfurous acid, monopropyl ester | HO—S(O)—OCH₂CH₂CH₃ |
| sulfurous acid, monopropyl ester, | Li•O—S(O)—OCH₂CH₂CH₃ |
| lithium salt | |
| sulfurous acid, monopropyl ester, | Na•O—S(O)—OCH₂CH₂CH₃ |
| sodium salt | |
| sulfurous acid, monopropyl ester, | 1/2 Mg•O—S(O)—OCH₂CH₂CH₃ |
| magnesium salt | |
| sulfurous acid, monophenyl ester | HO—S(O)—OPh |
| (phenyl hydrogen sulfite) | |
| sulfurous acid, monophenyl ester, | Na•O—S(O)—OPh |
| sodium salt (sodium phenyl sulfite) | |
| methanesulfinic acid (methyl- | HO—S(O)—CH₃ |
| sulfinic acid) | |
| sodium methanesulfinate | Na•O—S(O)—CH₃ |
| ethanesulfinic acid (ethylsulfinic | HO—S(O)—CH₂CH₃ |
| acid) | |
| sodium benzenesulfinate | Na•O—S(O)—Ph |
| methylamidosulfurous acid | HO—S(O)—NHCH₃ |
| sodium dialkylamidosulfinate | Na•O—S(O)—NRR′ |
| | R = R′ = Me or Et or (CH₂)₅ |

This list of compounds is exemplary only and is not intended to be limiting in any manner.

A representative synthesis of the monomethyl, monoethyl, and monoisopropyl ester sodium salts, AND THE synthesis of the corresponding dimethyl, diethyl, and di-isopropyl esters, is described by A. B. Foster et al. J. of the Chemical Soc. (1956) 2589-2592, incorporated herein by reference in its entirety. Synthesis of sodium dialkylamidosulfinate compounds (Na.O—S(O)—NRR′) wherein R═R′=Me or Et or (CH₂)₅ was reported by A. Blaschette and H. Safari, Z. fuer Naturforsch.(1970) 25, 319-320, also incorporated herein by reference in its entirety.

Some representative bis-substituted sulfur nucleophiles of formula I are listed in table 2:

| R1 | R2 | COMPOUND |
|---|---|---|
| OMe | OMe | Sulfurous acid dimethyl ester |
| OEt | OEt | Sulfurous acid diethyl ester |
| N(Me)₂ | OMe | Dimethyl-sulfinamic acid |
| | | methyl ester |
| N(Me)₂ | N(Me)₂ | |
| Me | Me | Methanesulfinylmethane |
| CMe₃ | NEt₂ | 2-Methyl-propane-2-sulfinic |
| | | acid diethylamide |

| | |
|---|---|
| —O—(CH₂)₂—O— | [1,3,2]Dioxathiolane 2-oxide |
| (forming a ring with S) | |
| —N(Et)-(CH₂)₂N(Et)- | 2,5-Diethyl- |
| (forming a ring with S) | [1,2,5]thiadiazolidine 1-oxide |

Of course these are non-limiting examples and are presented by way of illustration only. The table is not intended to limit the scope of the invention.

As discussed above, the substitutents of R₁ and R₂ may include various markers. These markers may be radio-labels, fluorescent moieties, or chemiluminescent moieties.

Radio labels are atoms or compounds that contain an atom that undergoes a process resulting in the emission of a photon, electron or other nuclear constituent, thus allowing their detection. Suitable radio-labels include, but are not limited to, ³H and ¹⁴C. These markers may be incorporated into any of the various substituents of R₁ and R₂.

The present invention is amenable to the use of a wide variety of fluorescent and chemiluninescent moieties, as are known in the art. Non-limiting examples of suitable fluorescent moieties include 6-carboxyfluorescein or 6-carboxytetramethylrhodamine. Suitable chemilumiscent moieties include, but are not limited to acridinium esters and derivatives thereof.

Those of ordinary skill in the art will readily recognize appropriate methods of making mono- and bis-substituted sulfur nucleophiles as well as salts and labeled versions thereof.

### Methods

According to some embodiments of the methods of the invention, a nucleic acid sample, containing a nucleic acid comprising at least one cytosine nucleobase is reacted with a nucleophilic organo-sulfur compound to facilitate conversion of cytosine to uracil for further assessment according to known techniques to determine methylation status. Such reactions may be performed by suitable adaptation of standard techniques for converting cytosine to uracil by using organo-sulfur compounds of the present invention in place of (or in addition to) bisulfite. For example, genomic DNA (1 microgram or less) is denatured for 15 to 30 minutes at 45° C. with NaOH (2M to 3M), followed by incubation with 0.1M hydroquinone and 3.6M sodium bisulfite (pH 5.0) at 55° C. for 12 hours or overnight. The DNA is then purified from the reaction mixture using standard miniprep columns, for example. For desulfuration, the purified DNA sample is resuspended in aqueous 0.25M NaOH (60 microliters) is incubated at 40° C. for 5-10 minutes. The desulfurated DNA can then be ethanol-precipitated and washed, followed by resuspension in water.

In some embodiments of the invention, a method for converting cytosine to uracil includes the step of reacting a nucleic acid comprising at least one cytosine nucleobase with a nucleophilic organo-sulfur compound, or a salt thereof, according to Formula I:

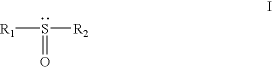

wherein R₁ and R₂ are as described above.

Reaction of the nucleophilic organo-sulfur compound with the cytosine containing nucleic acid results in specific conversion of cytosine, but not 5-methyl cytosine, to uracil. Upon conversion, known techniques, such as PCR, MSP, and other techniques, may be used to assess the methylation status of the sample.

In some embodiments, the nucleophilic organo-sulfur compound is a mono-substituted compound where R₂ is —OH and R₁ is as described above. Preferably, R₁ is selected from methyl or ethyl.

Again, upon completion of the reaction, known techniques may be used to assess the methylation status of the sample by analyzing conversion data.

Salts of the mono-substituted nucleophilic organo-sulfur compounds may also be employed. Particularly, the —OH group of R₂ is hydrolyzed to form an ionic bond with a cation. Preferred cations are lithium, sodium, ammonium or magnesium, and more particularly sodium, with the proviso that the compound is not a bisulfite compound.

In some embodiments, the nucleophilic compound is a bis-substituted compound where each of R₁ and R₂ is other than hydroxyl. R₁ is preferably methyl or ethyl and R₂ is preferably methyl or ethyl. As with the other embodiments herein, upon conversion, methylation status is assessed by known techniques.

Other embodiments employ the use of labels and detection of differing levels of labeling to determine methylation status. Labeling schemes in conjunction with existing single-molecule DNA-scanning procedures, or AFM (atomic force microscopy) technology, and other technologies, provides a powerful tool for discovery and analysis of, for example, methylated promoters of genes without the limitations associated with currently used RLGS methodology.

For example, either R₁ or R₂, or both, can include ³H or ¹⁴C labels for measurement of total 5-methyl cytosine vs. non-methylated cytosine content. Other radio-labels may be used as well. In this type of assay the DNA sample of interest (S) and a control sample (C) are separately reacted with the labeled reagent under identical reaction conditions. Following removal of excess labeled reagent, the difference in radioactivity of these two samples (R_{S} and R_{C}, respectively) provides a relative measure of 5-Methyl Cytosine content based on the expected differential reactivity of 5-methyl cytosine compared to Cytosine, namely, Cytosine reacts much more rapidly than 5-Methyl Cytosine. For example, if R_{S}=1000 counts/nucleotide-equivalent and R_{C}=2000 counts/nucleotide-equivalent, then sample of interest, S, is 50% methylated. Synthetic internal standards comprised of fully-methylated and non-methylated oligonucleotide sequences may be used as controls to normalize the raw data by correcting for low-levels of non-specific or incomplete reaction, respectively.

The labeling technique can be extended beyond radio-labels to marking with fluorescent moieties or moieties that allow chemiluminescence to be detected. Current optical methods, not employing differential labeling require use of sophisticated and expensive HPLC or CE equipment, and experienced operators. A significant advantage of differential labeling of methylated DNA using such reagents is that it provides a means of optically detecting sites of methylation such as CpG islands in promoter regions of genes. DNA reacted with fluorescent-labeled sulfur nucleophiles may be used with existing single-molecule DNA-scanning methods (S. Zhou et al. Genome res. (2003) 13, 2142-2151) to enable a method for genome-wide analysis of methylated promoters that does not require the use of radio labels and, moreover, is not limited to promoter regions having methylation-specific sites.

In this new approach, the elongated single-molecules of DNA are first imaged using YOYO-1 dye, as described (S. Zhou et al. Genome res. (2003) 13, 2142-2151), followed by removal of this dye and reaction with fluorescently labeled sulfur nucleophiles such that the DNA of interest is labeled in one color and the control DNA is labeled in a second color. The latter images are electronically subtracted such that 5-methyl cytosine is seen as a positive signal, which is then overlayed on the whole-genome map derived from the YOYO-1 data, as described (S. Zhou et al. Genome res. (2003) 13, 2142-2151.). In this manner, the methylated promoter regions of all genes are seen and identified by comparison with the relevant genome sequence.

Improved signal-to-noise ratios can be obtained by use of sulfur nucleophiles of formula I where R₁ and/or R₂ provide moieties that allow chemiluminescent imaging.

A fundamentally different scanning approach would use an adaptation of atomic force microscopy (K. Virnik et al. J. Mol. Biol. (2003) 334, 56-63.). The basic idea is to analyze differentially reacted DNA as an AFM-difference readout.

Analysis of total methylated DNA content by the above methods is relatively simple and does not require sophisticated, costly equipment operated by experts. These features are particularly advantageous in clinical settings.

In some embodiments, a method for converting cytosine to uracil includes the step of reacting a nucleic acid comprising at least one cytosine nucleobase with a mixture including a bisulfite ion and a nucleophilic organo-sulfur compound according to formula I above, or a salt thereof, according to Formula I. In this reaction, it is contemplated that the bisulfite ion reacts more quickly than the nucleophillic organo-sulfur compounds. The bisulfite is then displaced by the nucleophillic organo-sulfur compound. Methylation status may then be assessed according to known techniques. This approach may be used with labeled and unlabeled nucleophiles, but is particularly preferred with the labeled nucleophiles.

The examples described herein have been chosen to illustrate the invention, and are not intended to be limiting. Those reasonably skilled in the art will readily recognize additional embodiments that do not differ from the scope and spirit of the invention disclosed herein.

What is claimed is:

1. A method for converting cytosine to uracil in a nucleic acid comprising the steps of:
providing a nucleic acid comprising at least one cytosine nucleobase; and
reacting said nucleic acid with a nucleophilic organo-sulfur compound.

2. The method of claim 1 wherein said nucleophilic organo-sulfur compound is a compound of Formula I: 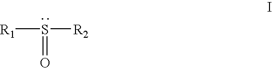
wherein
R₁ and R₂ are each independently selected from the group consisting of hydroxyl, alkyl, aryl, amino, alkoxy, and aryloxy, each of which may be optionally substituted; and
or R₁ and R₂ can be concatenated to form a 4-8 membered ring optionally having 1 or 2 additional hetero ring atoms selected from N, S, and O, wherein said ring can be optionally substituted with one or more substituents;
or a salt thereof.

3. The method of claim 2 wherein said amino of said R₁ and said R₂ has the formula NR₃R₄, and said alkoxy of said R₁ and said R₂ has the formula OR₅; and
wherein R₃, R₄ and R₅ are each independently selected from the group consisting of hydrogen, methyl, ethyl, propyl, isopropyl, 2-hydroxyethyl, and 2-methoxyethyl.

4. The method of claim 2 wherein said organo-sulfur compound is a salt of formula I where one of R₁ and R₂ forms an ionic bond with a cation selected from lithium, sodium, magnesium and ammonium.

5. The method of claim 2, wherein said reacting step is carried out with a mixture comprising a bisulfite ion and said nucleophillic organo-sulfur compound.

6. A method for assessing the methylation status of cytosine comprising the steps of:
providing a sample nucleic acid comprising at least one cytosine nucleobase of unknown methylation status; and
reacting said nucleic acid with a nucleophilic organo-sulfur compound comprising a radio-labeled substituent.

7. The method of claim 7 wherein said nucleophilic organo-sulfur compound is a compound of formula I: 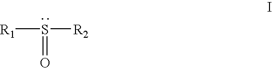
wherein
R₁ and R₂ are each independently selected from the group consisting of hydroxyl, alkyl, aryl, amino, alkoxy, and aryloxy, and a radiolabel substituent, wherein each of said alkyl, aryl, amino, alkoxy, and aryloxy can be optionally substituted;
wherein at least one of R₁ and R₂ comprises a radio-labeled substituent;
or a salt thereof.

8. The method of claim 8 further comprising the steps of:
providing a control nucleic acid comprising at least one cytosine nucleobase of known non-methylated status;
reacting said nucleic acid with the same said nucleophilic organo-sulfur compound; and
comparing the level of radioactivity of the sample and control to determine the relative content of methylated cytosine in the sample based on the rates of reaction of the methylated cytosine and unmethylated cytosine.

9. The method of claim 8 wherein said amino of said R₁ and said R₂ has the formula NR₃R₄, and said alkoxy of said R₁ and said R₂ has the formula OR₅; and
wherein R₃, R₄ and R₅ are each independently selected from the group consisting of hydrogen, methyl, ethyl, propyl, isopropyl, 2-hydroxyethyl, and 2-methoxyethyl.

10. The method of claim 8 wherein said organo-sulfur compound is a salt of formula I where one of R₁ and R₂ forms an ionic bond with a cation selected from lithium, sodium, magnesium and ammonium.

11. The method of claim 8, wherein said reacting step is carried out with a mixture comprising a bisulfite ion and said nucleophillic organo-sulfur compound.

12. A method for assessing the methylation status of cytosine comprising the steps of:
providing a sample nucleic acid comprising at least one cytosine nucleobase of unknown methylation status; and
reacting said nucleic acid with a nucleophilic organo-sulfur compound comprising a fluorescent or chemiluminescent moiety.

13. The method of claim 13 wherein said nucleophilic organo-sulfur compound is a compound of formula I: 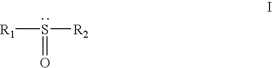
wherein
R₁ and R₂ are each independently selected from the group consisting of hydroxyl, alkyl, aryl, amino, alkoxy, and aryloxy, and a radiolabel substituent, wherein each of said alkyl, aryl, amino, alkoxy, and aryloxy can be optionally substituted;
wherein at least one of R₁ and R₂ comprises a fluorescent or chemiluminescent moiety;
or a salt thereof.

14. The method of claim 14 wherein said amino of said R₁ and said R₂ has the formula NR₃R₄, and said alkoxy of said R₁ and said R₂ has the formula OR₅; and
wherein R₃, R₄ and R₅ are each independently selected from the group consisting of hydrogen, methyl, ethyl, propyl, isopropyl, 2-hydroxyethyl, and 2-methoxyethyl.

15. The method of claim 14 wherein said organo-sulfur compound is a salt of formula I where one of R₁ and R₂ forms an ionic bond with a cation selected from lithium, sodium, magnesium and ammonium.

16. The method of claim 14 wherein said radio-labeled substituents comprises one of ³H and ¹⁴C.

17. The method of claim 14 further comprising the steps of:
providing a control nucleic acid comprising at least one cytosine nucleobase of known non-methylated status;
reacting said control nucleic acid with the same said nucleophilic organo-sulfur compound; and
detecting and comparing the level of optical activity of the sample and control to determine the relative content of methylated cytosine.

18. The method of claim 14, wherein said reacting step is carried out with a mixture comprising a bisulfite ion and said nucleophillic organo-sulfur compound.
